(19) United States
(12) Patent Application Publication
(10) Pub. No.: US 2009/0042779 A1
(43) Pub. Date: Feb. 12, 2009

(54) Purified Extract of an Alpha-Amylase Inhibitor From Phytoemagglutinin-Essentially Free Beans, Process for Its Extraction and Compositions Containing It

(76) Inventors: Roberto Bollini, Milan (IT); Francesca Sparvoli, Milan (IT)

Correspondence Address: NIXON & VANDERHYE, PC, 901 NORTH GLEBE ROAD, 11TH FLOOR, ARLINGTON, VA, 22203, US

(21) Appl. No.: 11/547,507

(22) PCT Filed: Mar. 25, 2005
(86) PCT No.: PCT/IB05/00779

§ 371 Date: Dec. 15, 2006

(30) Foreign Application Priority Data
Mar. 30, 2004 (IT) MI2004A000619

(51) Int. Cl. C07K 14/415; A61K 38/16; A61P 3/00; A61P 3/06

(52) U.S. Cl. 514/12; 530/370

(57) ABSTRACT

The invention relates to the use of beans essentially free from phytohemagglutinin for extracting the alpha-amylase inhibitor (hereinafter also “phaseolamin”), the extract enriched in phaseolamin, the highly purified extract, and also the dietetic and pharmaceutical compositions that contain it; the invention also relates to a process for preparing the above extract and for the immobilisation of phaseolamin on an inert solid support.

The present invention relates to the use of beans essentially free from phytohemagglutinin for extracting the alpha-amylase inhibitor (hereinafter also “phaseolamin”), the extract enriched in phaseolamin, the highly purified extract, and the dietetic and pharmaceutical compositions that contain it.

The invention also relates to a process for preparing the above extract and also the immobilisation of phaseolamin on an inert solid support. It has been known for a long time that bean seeds (Phaseolus vulgaris) contain an alpha-amylase inhibitor (phaseolamin) that is able to inhibit the digestion of starch and thus prevent the consequent formation of the simplest sugars which can be easily absorbed by the intestinal membranes.

Phaseolamin is a protein which is synthesised in the form of a precursor which consists of a limited number of isoforms of about 40 kDa having a different degree of glycosylation. Each isoform of the precursor is subsequently modified by means of an endoproteolitic cleavage in a central zone of the amino acid sequence; in this way the two characteristic components (subunit α and subunit β) of the mature form accumulated in the seed are generated (Santino A., Daminati M. G., Vitale A., Bollini, R. (1992) The α-amylase inhibitor of bean seed: two-step proteolytic maturation in the protein storage vacuoles of the developing cotyledon. Physiologia Plantarum 85, 425-432). In its native form, phaseolamin is an oligomer with composition α₂β₂ in which the subunits are associated in a non covalent way. In some cases there is also a subunit of about 30 kDa (component δ) and the three subunits α, β and δ may be associated with each other in various combinations to give the oligomers δ₂, δαβ and α₂β₂.

Untreated bean extracts or bean protein concentrates have been present for some time on the market which, as well as phaseolamin, also contain the other protein major components of the seed. Particularly important is the presence of phytohemagglutinin (PHA), a highly toxic protein if consumed in native conditions such as those of the protein contained in the raw seed and in dietetic products on the market.

This phytohemagglutinin, or lectin, is deactivated by the heat during the cooking the beans, but this heat also deactivates phaseolamin. It is thus clear that the alpha-amylase inhibiting effects necessary to block the digestion of starch may be obtained only by assuming bean extracts in native conditions, that is non denatured, which however would also involve the consumption of toxic phytohemagglutinin. EP-1213972 claims a dietetic composition composed of an association of an alpha-amylase inhibitor and a compound chosen from inulin, fructooligosaccharides and eventually a fat absorption inhibitor. As a particularly preferred alpha-amylase inhibitor it mentions phaseolamin, obtained by Leuven Bioproducts by extraction from Phaseolus vulgaris, referred to in the document as “Phaseolamin™”. From the biochemical description of the molecule (pure L4 and isoform of PHA-E) it may however be deduced that the protein concerned is not phaseolamin but rather phytohemagglutinin (PHA) (see for comparison Bollini, R., Chrispeels, M. J. (1978) Characterization and subcellular localization of vicilin and phytohemagglutinin, the two major reserve proteins of Phaseolus vulgaris L. Planta 142, 291-298). It is known that phaseolamin and phytohemagglutinin originated from the evolution of a common ancestral precursor belonging to the family of lectins of leguminosoe (Lioi, L., Sparvoli F., Galasso, I., Lanave C., Bollini R. (2003) Lectin-related resistance factors against bruchids evolved through a number of duplication events. Theoretic and Applied Genetics 107, 814-822). The molecules of the two proteins, which are the result of genic duplications and punctiform mutations of the ancestral sequence, though showing a different biological activity, have maintained very similar chemical and physical properties. Consequently, in the extraction of phaseolamin from the Phaseolus vulgaris, also the toxic phytohemagglutinin is solubilised, and the two proteins, which constitute a principal proteic component of the extract, are then extremely difficult to separate.

It is therefore necessary to have bean extracts enriched in phaseolamin but free from phytohemagglutinin, advantageously obtained with simple process which give good yields.

The hypothesis has therefore been expressed of using beans essentially free from phytohemagglutinin, for example with the characteristic “lack of phytohemagglutinin” (hereinafter also only “PHA-null beans” or “phytohemagglutinin-null”) to prepare extracts rich in phaseolamin and free from the toxic component.

In particular, to eliminate the presence of the toxic protein PHA in the above extracts in an easy and economically convenient way, it has been thought to develop beans which, genetically, were not able to accumulate phytohemagglutinin in the seed. Secondly, to make the process even more convenient, it has been thought to give these beans two important genetic characteristics which they did not possess before: a) high productive capacity; b) dwarf plant size. The presence of the latter genetic character, in particular, allows mechanization of all the stages of plant cultivation, with the result of enormously reducing the costs of seed production.

In this case the seed produced is more easily digestible and is an innovative source from which to extract bioactive molecules (Bollini R., Carnovale, E., Campion, B. Removal of antinutritional factors from bean Phaseolus vulgaris seeds Biotechnol. Agron. Soc. Environ., 1999, 3(4):217-219).

The presence of phytohemagglutinin may be advantageously detected using its capacity of agglutinating eryhtrocytes in vitro. Untreated extracts of the recurrent parent (Taylor's Horticultural) and of commercial varieties show agglutinating activity even in high dilutions (Table 1). This action is almost totally eliminated by cooking, mimed by denaturing the extracts for 15 min at 100° C. On the other hand the erythroagglutinating activity possibly present in the extract of the modified seeds with the “phytohemagglutinin-null” genetic characteristic is lower than the sensitivity limit of the test carried out in the same experimental conditions and is at any rate lower than the residual activity present in the denatured extracts.

Below, Table 1 shows the hemoagglutinating activity present in 50 μl of extract, corresponding to 1 mg of flour, expressed as the inverse of the last serial dilution in which can be visually detected the agglutination of rabbit erythrocytes present in 50 μl of a 0.5% suspension. The error is of +/1 dilution.

TABLE 1

| | Agglutinating activity |
|---|---|
| Bean variety | of untreated extracts |
| PHA-free variety | Not detectable |
| Taylor's Horticultural | 512 |
| Denatured Taylor's Horticultural | 4 |
| Billò | 1024 |
| Denatured Billò | 4 |
| Giulia | 1024 |
| Denatured Giulia | 2 |
| Greensleeves | 512 |
| Denatured Greensleeves | 1 |

The inventors therefore prepared a process for extraction from beans essentially free from phytohemagglutinin that supplies a highly purified phaseolamin extract, which is therefore safe and suitable for consumption by man and animals

The bean seeds essentially free from phytohemagglutinin used as the starting material for preparing the extract in the invention are preferably from modified beans with the “phytohemagglutinin-null” characteristic. The “phytohemagglutinin-null” genetic characteristic (Vitale, A., Ceriotti, A., Bollini, R. (1985) Molecular analysis of a phytohemagglutinin-defective cultivar of Phaseolus vulgaris L. Planta 166, 201-207) was associated with the determined vegetative habitus (dwarf plant) by means of crossing and selecting cycles in an agronomically valid dwarf variety. Some of the lines obtained during the intermediate phase of the selection cycle were agronomically tested, showing that they had a good productive capacity (Confalonieri et al., Plant Breeding, 1992, 109:329-334).

The selection of superior lines continued until the homozygote condition of the “phytohemagglutinin-free” character was achieved (lec/lec), in order to obtain a source of phaseolamin with a high productive yield. Said beans are also the object of a jointly pending patent application for a plant variety. As an alternative it is possible to lower or block the synthesis of phytohemagglutinin by means of genetic transformation. The genetic transformation of the bean, though with low yields, has recently been described by Aragao F J L, et al (1996) Inheritance of foreign genes in transgenic bean (Phaseolus vulgaris L.) co-transformed via particle bombardment. Theoretic and Applied Genetics 93, 142-150; Genga, A., Allavena, A., Ceriotti, A., Bollini, R. (1990) Towards genetic transformation of bean by Agrobacterium tumefaciens. Acta Horticolturae 280, 527-536.

So, according to one of its aspects, the invention relates to a process for the preparation of a proteic extract from essentially phytohemagglutinin-free (PHA-null) beans which comprises extracting the flour of said beans with distilled water at environment temperature, acidifying it to a pH of about 5, heating and separating the soluble phase containing the phaseolamin from the insoluble precipitate.

The extract thus obtained, enriched in the phaseolamin content, may be further purified, for example by fractionated precipitation with ammonium sulphate and by means of ion exchange chromatography on resins, and subsequently subjected to optional desalification. The phaseolamin extract obtained after the phases of purification and desalification of the invention is extremely pure and free from contaminating toxic substances, for example phytohemagglutinin. According to an advantageous embodiment, the present invention relates to a process for preparing a proteic extract from essentially phytohemagglutinin-free bean seeds and which comprises the following steps:
(i) extracting flour from said bean seeds with water;
(ii) acidifying the extract to an acidic pH, about 5, heating from 50 to 80° C. and separating the precipitate from the liquid phase;
(iii) precipitating the phaseolamin by saturating the liquid phase up to 60% to 70% with ammonium sulphate and recovering the precipitate, for example by centrifugation;
(iv) purifying the liquid phase of step (ii) or the precipitate of step (iii) by ion exchange chromatography on resin; and
(v) desalifying the phaseolamin thus obtained.

In step (i) of the process it is preferable to use deionised water and a flour/solvent ratio from 1/10 to 1/20, advantageously from 1/13 to 1/18, even better with a ratio of about 1/15 (p/p). A ratio between flour and water close to 1/10 allows efficacious extraction but gives a very dense suspension in which it is difficult to separate the liquid phase containing the phaseolamin (and the other soluble proteins of the seed) from the insoluble residue. Ratios higher than 1/15 do not significantly increase the yield of inhibitor but they produce greater volumes to be treated.

An intermediate flour-solvent ratio, for example 1:15, was particularly advantageous although other relative quantities may however be used for extraction.

Extraction with water, preferably distilled or deionised water, is advantageously carried out by keeping the suspension stirred at room temperature and it is generally completed in about two hours, although it may be protracted for longer periods. A second extraction with a smaller volume of water may lead to an increase in the yield of phaseolamin.

In step (ii) acidification may be carried out with any mineral acid, advantageously with diluted hydrochloric acid 1 N, better if 2 N. Heating, for example at 60-70° C., should preferably be kept up for a short period, around 15 minutes. The separation of the precipitate may be carried out with any means known to the skilled in the art, for example by centrifugation.

In step (iii) the supernatant is advantageously brought to 40% of saturation with ammonium sulphate, keeping the pH around 7.0 and the precipitate is eliminated by centrifugation; the phaseolamin is then precipitated with a further addition of salt up to 65% saturation and recovered, for example by centrifugation. The phaseolamin is thus strongly enriched and may be further purified by ion exchange chromatography which allows efficacious separation of the proteins. In step (iv) the purification is obtained for example by anion exchange chromatography according to the known techniques. The use of a resin of the Sepabeads® series (Mitsubishi Chemical Co.) is advantageous, preferably of the Sepabeads FP-DA type.

Advantageously, the aqueous phase of step (ii) is loaded in a column, just as it is or, more advantageously, brought to a neutral pH value, preferably pH 7.5, with a diluted alkaline solution (for example with NaOH IM), or the precipitate of step (iii) dissolved in the buffer in which the resin is balanced, and is eluted with a solution of NaCl, or said preparations are loaded in NaCl around 0.2 M and eluted with a buffer, advantageously a buffer 1 M NaCl. The use of concentrations of NaCl around 0.2 M directly during loading or before the application of more concentrated solutions of NaCl allows the recovery of different proteic fractions while for the elution of phaseolamin it is necessary to increase the concentration to about 1 M NaCl.

Desalification may be carried out according to the process well known to the skilled in the art, for example by ultrafiltration on a membrane or by chromatography on resin with hydrophobic interaction, advantageously on Sepabeads^{R} FP-BU, or by precipitation with two volumes of acetone.

Details of the steps from (i) to (v) described above are given in the experimental section. The extraction and purification process may be followed by mean of SDS-PAGE analysis.

The final product of the extractions and purifications is essentially composed of phaseolamin and totally free from contamination by phytohemagglutinin.

The solid phaseolamin is obtained from the solutions that contain it by precipitation, for example with two volumes of acetone, or by freeze-drying.

Activity = Unit of α-amylase from human saliva inhibited in 30′

| | mg | Total | | Activity/ | |
|---|---|---|---|---|---|
| | protein/ | Activity/ | | mg | |
| | g flour | g flour | yield | protein | x |
| Untreated extract | 78 | 13.000 | 100% | 167 | |
| Step I | | | | | |
| Acid supernatant | 28 | 10.400 | 80% | 370 | 2.2 |
| Step II | | | | | |
| Pellet 65% | 15.6 | 8.450 | 65% | 540 | 3.2 |
| Step III | | | | | |
| Pure phaseolamin, | | | | >2400 | >14.4 |
| Step IV | | | | | |

(x = multiplication factor of the activity of the untreated extract)

The extract containing phaseolamin and the purified phaseolamin obtained with the process according to the invention represents a further aspect of the invention.

The phaseolamin obtained with the extraction process in the invention is composed of two subunits α and β of about 15-20 kDa and of the component δ of about 30 kDa.

For use in the dietetic and/or pharmaceutical field the extract or the phaseolamin in the invention are preferably administered in dietetic and/or pharmaceutical compositions, in a free form or adsorbed on a solid support, according to the techniques known to the experts in the sector, for example on sepharose or another compatible support. According to another of its aspects, the invention relates to compositions for dietetic use comprising the phaseolamin obtained with the process described above.

These dietetic compositions are intended to facilitate weight loss thanks to the inhibition of starch degradation by alpha-amylase and they are therefore particularly suitable as adjuvants in low-calorie diets, in the treatment of conditions of overweight and obesity.

The compositions of the invention are more generally useful when it is desired to limit the absorption of the sugars taken with the diet and are for example a useful dietetic aid in diabetic subjects.

According to another of its aspects, the invention relates to the use of essentially phytohemagglutinin-free beans in the preparation of dietetic extracts enriched with alpha-amylase inhibitors.

According to a particularly preferred aspect, the invention relates to the use of modified beans with the “phytohemagglutinin-free” characteristic in the preparation of dietetic extracts enriched with alpha-amylase inhibitors.

In particular, the invention also relates to the use of essentially phytohemagglutinin-free beans, for example of modified beans with the “phytohemagglutinin-null” characteristic, in preparing dietetic compositions for inhibiting starch absorption and intended for reducing body weight.

With respect to the dietetic compositions on the market, the compositions of the invention are safer and more efficacious, as they do not contain even any traces of phytohemagglutinin, thanks to the use of original bean seeds modified in such a way as not to express that substance.

The compositions of the invention are intended to be taken orally. These compositions may for example be in the form of tablets, capsules, granules, powders, etc.

In these compositions, the phaseolamin extract obtained from essentially phytohemagglutinin-free beans with the process according to the invention is mixed with the excipients for oral use conventionally used in the food and/or pharmaceutical technique, for example with gelatine, lactose, magnesium stearate, talc, gum arabic, or similar, well known to the skilled in the art.

Some preferred compositions of the invention comprise enriched extracts or essentially pure phaseolamin in concentrations ranging advantageously from about 5% to 100% weight/total weight of the composition. Different concentrations of phaseolamin may however be contemplated depending on the type of formulation or in general on the requirements of the subject to be treated.

The compositions may be formulated in dosing units comprising preferably from 5 to 1,000 mg of phaseolamin, advantageously from 10 to 500 mg, for example from 50 to 350 mg. These compositions will be administered as necessary.

In the compositions of the invention, the phaseolamin extract may optionally be mixed with other active principles, for example with other substances that act on the reduction of food absorption, or that are able to activate the metabolism.

Examples of compositions according to the invention will be supplied in the following experimental section.

### DESCRIPTION OF THE FIGURES

FIG. 1: SDS-PAGE analysis of untreated extracts from seeds of commercial varieties and from the modified variety. Row 1: recurrent parental cultivar Taylor's Horticultural (Asgrow); row 2: new PHA-free variety; row 3: local cultivar Billó; variety Giulia (ISPORT).

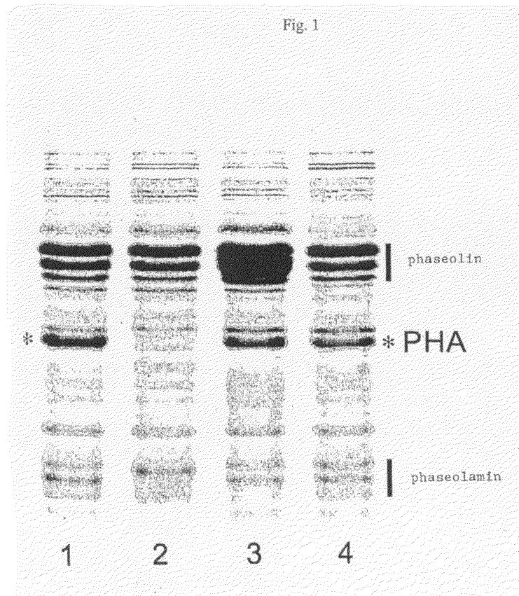

FIG. 2: SDS-PAGE analysis of an untreated extract of the PHA-free variety (row 1) and of two independent preparations of extracts as in point II of Example 1 (after acidification and heating) (rows 2 and 3).

FIG. 3: A) typical chromatogram of the anion exchange purification on resin of the extract from phase II of Example 1. The phaseolamin present is highly purified in the eluted peak with a high concentration of NaCl. The presence of larger quantities of subunit δ in the head of the peak (insert 2) with respect to the tail (insert 3), suggests that the oligomers containing the subunit δ are preferentially eluted before those with only the subunits α and β. B) Comparison between the composition of the extract obtained in point II and the essentially pure phaseolamin isolated from point III (Example 1).

FIG. 4: SDS-PAGE analysis of the proteins precipitated from point II with 40% (row 1) and 65% (row 2) saturation of ammonium sulphate.

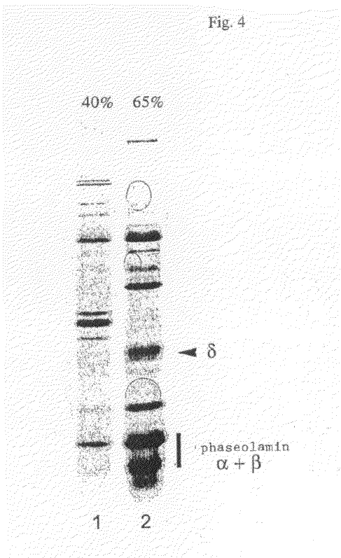

FIG. 5: comparison between the n-terminal sequence of the subunit δ (experimentally obtained) with the sequences of two variants of phaseolamin and of the subunit L of phytohemagglutinin (present in the literature).

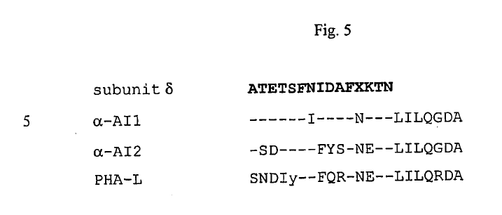

### EXPERIMENTAL PART

### Example 1

### Preparation of the Phaseolamin Extract

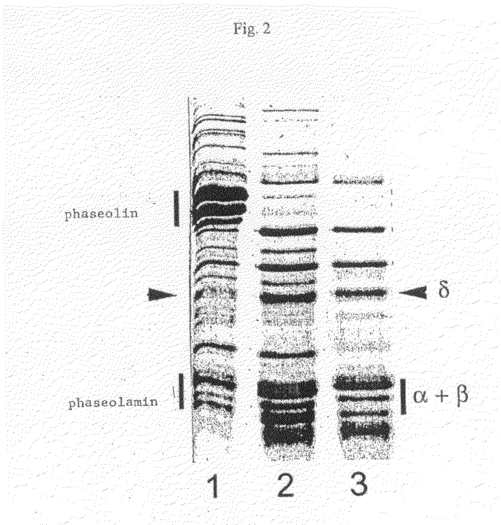

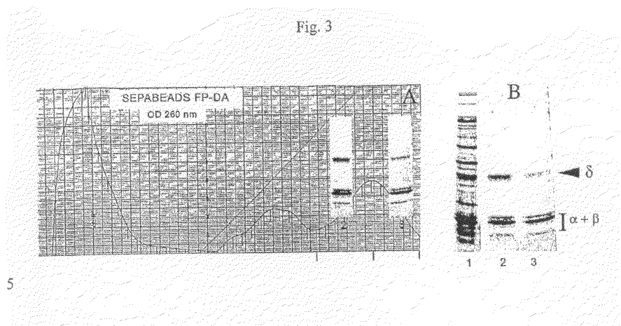

I) The protein is efficiently extracted by suspending bean flour directly with deionised water in a ratio of 1:15 (p/p) and keeping it stirred for two hours at room temperature. The insoluble residue is separated from the aqueous fraction by centrifugation at 10,000×g for 30 min.
II) The untreated extract is acidified to pH 4.5 by adding HCl 2N. The precipitation of phaseolin and of other minor proteins instable to heat, as well as to the denaturizing of the endogenous amylase, is improved by heating the extract for 15 min at 70° C. while stirring. The extract is then brought back to room temperature and the separation of the coagulated insoluble material is carried out as in point I). The extract is highly enriched in phaseolamin, which now represents the major proteic component (FIG. 2, compare row 1 and rows 2-4).
III) The phaseolamin may be further enriched by means of fractionated precipitation with ammonium sulphate. The addition of salt to the extract in point II), up to 40% saturation, keeping the pH around 7.0, causes the formation of a precipitate which is removed by centrifugation. A further addition of ammonium sulphate up to 65% saturation results in the total precipitation of the phaseolamin and of the other contaminating proteins. The precipitate is recovered by centrifugation.
IV) (a) A further purification of the phaseolamin is obtained by ion exchange chromatography on resin. The extract in point II) or the precipitate in point iii) are brought to 25 mM NaCl, 10 mM sodium phosphate, pH 7.5 and loaded on Sephabeads FP-DA anion exchange resin balanced in the same buffer. The resin is then washed with the load buffer to eliminate the non bound proteins and the chromatography is developed by applying a gradient 25 mM—1 M NaCl in 10 mM sodium phosphate, pH 7.5. The phaseolamin is present in the last peak of proteins, eluted in concentrations of NaCl higher than 0.2 M (FIG. 3). (b) Alternatively, the preparations mentioned in point II) and iii) are loaded directly in NaCl 0.2 M, 10 mM sodium phosphate, pH 7.5 and, after washing of the column with a volume of the same buffer, the phaseolamin is eluted by directly applying a buffer 1 M NaCl, 10 mM sodium phosphate, pH 7.5.
V) The different preparations of alpha-amylase inhibitor as in point iv) are completely desalified by passing on a SEPABEADS FP-BU column with hydrophobic interaction.

The fractions coming from the chromatographs are loaded, in a column, on SEPABEADS FP-BU resin balanced in 1 M NaCl, 25 mM phosphate pH 7.5 and the inhibitor is eluted by washing the same with deionised water.

The extraction process was followed by means of analysis on polyacrylamide gel in denaturing conditions as described previously (Confalonieri et al., Plant Breeding, 1991, 109:329-334) and the presence of the inhibitor in the chromatogram fractions was confirmed by dosing of the activity in vitro.

### Example 2

### Dietetic Composition with a Base of Phaseolamin Extract

The following components are mixed:

Phaseolamin obtained according to the example 1)

Excipients and other active principles

1. A process for preparing an alpha-amylase inhibitor of the bean characterised by extracting flour from essentially phytohemagglutinin-free bean seeds with water at room temperature, acidifying to a pH of about 5, heating and separating the aqueous phase containing the phaseolamin.

2. Process according to claim 1 characterised in that the phaseolamin thus extracted is purified by means of ion exchange chromatographies on columns and then desalified.

3. Process according to claim 1 characterised by:
(i) extracting a flour from modified bean seeds with water;
(ii) acidifying the extract to an acidic pH, about 5, heating from 50 to 80° C. and separating the precipitate from the liquid phase;
(iii) precipitating the proteins with ammonium sulphate;
(iv) purifying the liquid phase and the precipitate of the previous steps by ion exchange chromatography on resin;
(v) desalifying the phaseolamin thus obtained.

4. Process according to claim 3 characterised in that the flour/water ratio is about 1/15 (p/p).

5. Process according to claim 3 characterised in that acidification is obtained by adding a mineral acid.

6. Process according to claim 5 characterised in that said mineral acid is hydrochloric acid.

7. Process according to claim 3 characterised in that in step (iv) purification is carried out on anion exchange resin.

8. Process according to claim 3 characterised in that in step (v) desalification is carried out by chromatography on a hydrophobic interaction resin or by ultrafiltration on a membrane.

9. Process according to any one of the previous claims, characterised in that said bean seeds essentially free from phytohemagglutinin are modified seeds of beans with the “phytohemagglutinin-null” characteristic.

10. Process according to any one of the previous claims, characterised in that said bean seeds essentially free from phytohemagglutinin are bean seeds in which the synthesis of phytohemagglutinin has been blocked by genetic transformation.

11. Phaseolamin obtained with the process in the claims from 1 to 10.

12. Phaseolamin according to claim 11 adsorbed on a solid support.

13. A dietetic and/or pharmaceutical composition comprising as active principle the phaseolamin obtained with the process in the claims from 1 to 10.

14. Composition according to claim 13 in the form of tablets, capsules, granulates, powders.

15. Composition according to claim 13 or 14 which also comprises one or more other active principles useful for reducing body weight.

16. Composition according to the claims from 13 to 15 intended for the treatment of excess weight and obesity.

17. Use of the phaseolamin of claim 11 or 12 for preparing a drug or a dietetic product intended to control or to reduce body weight, to prevent and/or treat obesity and to decrease sugar absorption.

18. Use of modified bean seeds with the phytohemagglutinin-null characteristic to prepare dietetic extracts enriched in alpha-amylase inhibitors.

19. Use according to claim 18 for preparing dietetic extracts enriched in phaseolamin.
